(19) United States
(12) Patent Application Publication
(10) Pub. No.: US 2003/0060444 A1
(43) Pub. Date: Mar. 27, 2003

(54) Cell activation process and reagents therefor

(75) Inventors: Helene Margaret Finney, Berkshire (GB); Alastair David Griffiths Lawson, Hants (GB); Andrew Neil Charles Weir, Berkshire (GB)

Correspondence Address: SIGNATURE OF PRACTITIONER, P.O. Box 9169, Boston, MA, 02209, US

(73) Assignee: CELLTECH THERAPEUTICS, LTD.

(21) Appl. No.: 10/280,596

(22) Filed: Oct. 24, 2002

Related U.S. Application Data
(63) Continuation of application No. 09/446,529, filed on May 19, 2000, now abandoned.
Application No. 09/446,529 is a 371 of international application No. PCT/GB98/01842, filed on Jun. 24, 1998.

(30) Foreign Application Priority Data
Jun. 25, 1997 (GB) 9713473.8

(51) Int. Cl. A61K 48/00; C12P 21/02; C12N 5/08; A61K 38/20

(52) U.S. Cl. 514/44; 435/69.5; 435/320.1; 435/366; 424/85.1; 424/85.2

(57) ABSTRACT

A cell activation process is described in which an effector cell is transformed with DNA coding for a chimeric receptor containing two or more different cytoplasmic signalling components. At least one of the cytoplasmic signalling components is derived from all or part of a tetraspan-transmembrane protein, CD43, CD6, a mannose, U17, IL-12 or complement receptor, an integrin-associated protein, or a γ-chain associated with a cytokine receptor. The activated cell may be of use in medicine for example in the treatment of diseases such as cancer.

This invention relates to a process for activating cells, a DNA delivery system for achieving cell activation and the use of activated cells in medicine.

The natural T-cell receptor is a complex association of polypeptide chains comprising antigen binding, transmembrane and cytoplasmic components. Binding of antigen to the receptor in the correct context triggers a series of intracellular events leading to activation of the T-cell and for example destruction of the antigen presenting target cell. Before recognition of the antigen can take place, the antigen must be presented in association with MHC molecules.

It would be highly desirable if this requirement for MHC could be bypassed by engineering T-cells to become active on binding ligands other than a natural MHC-presented antigen. This would provide a means of avoiding the variability between individuals associated with MHC presentation and would also permit the targeting of more highly expressed surface antigens thereby increasing the efficacy of lymphocyte mediated therapy, for example in tumour therapy.

Chimeric receptors have been designed to target T-cells to cells expressing antigen on their cell surface. Such recombinant chimeric receptors include chimeras containing binding domains from antibodies and intracellular signalling domains from the T-cell receptor, termed ‘T-bodies’ [see for example Published International Patent Specifications Nos. WO 92/10591, WO 92/15322, WO 93/19163 and WO 95/02686].

The recombinant chimeric receptors described in the art are composed of a ligand binding component, a transmembrane component and a cytoplasmic component. It has been found however, that transfection of T-cells with these recombinant chimeric receptors does not result in acceptable levels of T-cell activation upon antigen binding unless the T-cell is also co-stimulated by, for example, treatment with high levels of interleukin 2 [II-2]. The need for co-stimulation makes the method suitable principally for ex-vivo treatment of patients. This is a lengthy and complicated procedure.

In our International Patent No. WO 97/23613 we provide an alternative to the present ex-vivo approach which achieves improved ex-vivo activation without the need for addition of costimulating agents such as II-2. The invention described there also advantageously provides successful in-vivo redirection and activation of T-cells, particularly in response to a single type of extracellular interaction.

Essentially the invention described in International Patent Specification No. WO 97/23613 provides an effector cell which has been transformed with DNA coding for a chimeric receptor. The chimeric receptor contains two or more different signalling cytoplasmic components which are not naturally linked and which advantageously are chosen to act together cooperatively to produce improved activation of the cell. DNA coding for such recombinant chimeric receptors may be introduced into T-cells or other effector cells in-vivo and/or ex-vivo. Subsequent binding of an effector cell expressing one or more of these chimeric receptors to a target cell elicits signal transduction leading to activation of the effector cell in a process involving clustering or dimerisation of chimeric receptors or allosteric changes in the chimeric receptor or another mechanism for receptor-triggering.

We have now developed the invention described in International Patent Specification No. WO 97/23613 to provide a series of chimeric receptors which advantageously can be used to precisely tailor the activation or response of effector cells. This can be achieved by making use of receptors with particular co-stimulatory signalling domains.

Thus according to one aspect of the invention we provide a method of activating a cell as a result of one type of extracellular interaction between said first cell and a molecule associated with a second target cell in which said first cell is provided with a DNA delivery system comprising DNA coding for one or more recombinant chimeric receptors comprising two or more different cytoplasmic signalling components, wherein said cytoplasmic components are not naturally linked, and at least one is derived from a membrane spanning polypeptide, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor.

The DNA coding for the chimeric receptor(s) is arranged such that when it is expressed, and on the extracellular interaction between the cell and a second target cell, a signal is transduced via the cytoplasmic signalling components to two or more different intracellular signalling messengers. This results in activation of the cell and elicits a biological response to the target cell. As used herein, cell activation means activation of one or more signal transduction pathways. This may be evidenced by an increase in cell proliferation; expression of cytokines with, for example pro or anti-inflammatory responses; stimulation of cytolytic activity, differentiation or other effector functions; antibody secretion; phagocytosis; tumour infiltration and/or increased adhesion.

The cytoplasmic signalling components, including the co-stimulatory signalling domains, are preferably selected such that they are capable of acting together cooperatively. They are “not naturally linked”, which term is used herein to denote cytoplasmic signalling components which in nature are not connected to each other on a single polypeptide chain. Preferably the cytoplasmic signalling components are those which are capable of linking to and/or generating a response in one or more cytoskeletal components of the cell in which they are expressed. Particularly useful signalling components include those described hereinafter in relation to other aspects of the invention.

In addition to the cytoplasmic signalling components each recombinant chimeric receptor preferably comprises a binding component capable of recognising a cell surface molecule on a target cell, and a transmembrane component. The DNA coding for these components will additionally code for a signal peptide to ensure that the chimeric receptor(s) once expressed will be directed to the cell surface membrane. All the components may be coded for by a single DNA coding sequence.

Alternatively, each cytoplasmic signalling component may be coded for by two or more separate DNA coding sequences. In this instance each DNA coding sequence may also code for a signal peptide, a transmembrane component and/or a binding component. The binding components may be different, but will generally all be capable of participating in the same type of extracellular event, for example by binding to the same molecule associated with the target cell. In one preference the binding components are the same.

In some of the applications described hereinafter, for example where the binding component is an antibody or an antibody fragment, the DNA coding for the chimeric receptor may comprise two separate DNA coding sequences, one sequence for example coding for part of the binding component [in the case of an antibody for example a V_{H} component] linked to the signal peptide, transmembrane and cytoplasmic signalling component(s), and the second sequence coding for the remainder of the binding component [for example a V_{L} component in the example given].

In order to activate a desired cell the DNA coding for the chimeric receptor will first need to be delivered to the cell. Thus according to a second aspect of the invention we provide a DNA delivery system comprising DNA in association with a carrier said DNA coding for a recombinant chimeric receptor capable of one type of extracellular interaction and comprising two or more different cytoplasmic signalling components which are not naturally linked, and wherein at least one of said cytoplasmic components is derived from a membrane spanning polypeptide characterised in that at least one of said cytoplasmic signalling components is a co-stimulatory signalling domain derived from all or part of a tetra-span-transmembrane protein, CD9, CD43, CD6, a mannose, IL7, IL-12 or complement receptor, an integrin-associated protein, or a γ-chain associated with a cytokine receptor.

In this aspect of the invention the chimeric receptor may be coded for by a single DNA coding sequence, coding in particular for the two or more different cytoplasmic signalling components. Thus in one preference the invention provides a DNA delivery system comprising DNA in association with a carrier said DNA coding for a recombinant chimeric receptor wherein said DNA codes in reading frame for:

i) a signal peptide component;

ii) a binding component capable of recognising a cell surface molecule on a target cell;

iii) a transmembrane component;

iv) two or more different cytoplasmic signalling components which are not naturally linked, and wherein at least one of said cytoplasmic components is derived from a membrane spanning polypeptide, and optionally

v) one or more spacer regions linking any two or more of said i) to iv) components,

characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein or a γ-chain associated with a cytokine receptor.

The components of the recombinant chimeric receptor are operatively linked such that the signalling cytoplasmic components are functional in transducing a signal resulting in activation of one or more messenger systems as a result of recognition of a cell surface molecule on a target cell by the binding component.

Two or more of the components may be linked by one or more spacer regions. The spacer regions may function to facilitate the components adopting the correct conformation for biological activity. The use of a spacer region to link the transmembrane component iii) and the binding component ii) is particularly advantageous.

The spacer regions may for example comprise up to 300 amino acids and preferably 20 to 100 amino acids and most preferably 25 to 50 amino acids.

Spacers may be derived from all or part of naturally occurring molecules such as from all or part of the extracellular region of CD8, CD4 or CD28; or all or part of an antibody constant region, including the hinge region. All or part of natural spacing components between functional parts of intracellular signalling molecules for example spacers between ITAMS (immunoreceptor tyrosine based activation motifs) may also be used. Alternatively the spacer may be a non-naturally occurring sequence.

The binding component ii) may be any molecule capable of interacting with cell surface molecules and may be chosen to recognise a surface marker expressed on cells associated with a disease state such as for example those associated with virally infected cells; bacterially infected cells; cancer cells, such as the bombesin receptor expressed on lung tumour cells, carcinoembryonic antigen, polymorphic epithelial mucin, and CD33; peptide hormones, adhesion molecules, inflammatory cells present in autoimmune disease, or a T-cell receptor or antigen giving rise to autoimmunity.

Suitable binding components for use in the chimeric receptors of the invention also include all or part of receptors associated with binding to cell surface associated molecules; the T-cell receptor; CD4; CD8; CD28; cytokine receptors e.g. an interleukin receptor, TNF receptor, or interferon receptor e.g. γ-IFN; receptors for colony stimulating factors e.g. GMCSF; antibodies and antigen binding fragments thereof including for example Fab, Fab′, F(ab′)₂, single chain Fv, Fv, and V_{H} or V_{L} components which may be in association with C_{H} and C_{L} domains. The antibodies or fragments may be murine, human, chimeric or engineered human antibodies and fragments. As used herein the term engineered human antibody or fragment is intended to mean an antibody or fragment which has one or more CDR's and one or more framework residues derived from one antibody, e.g. a murine antibody embedded in an otherwise human framework. Such antibodies are well known and may be prepared by a number of methods for example as described in International Patent Specification No. WO91/09967.

Particularly useful binding components include Fab′ fragments or, especially, single chain Fv fragments.

When the binding component is an antibody or antibody fragment other than a single chain Fv or V_{H} or V_{L} component which contains separate binding chains it will be necessary to include a second separate DNA coding sequence in the delivery system according to the invention to code for the second binding chain. In this instance the first DNA sequence containing the cytoplasmic signalling components and one chain of the antibody or fragment will be coexpressed with the second DNA sequence coding for a signal peptide and the second chain of the antibody or fragment so that assembly of the antibody binding component can occur.

Transmembrane components iii) may be derived from a wide variety of sources such as all or part of the alpha, beta or zeta chain of the T-cell receptor, CD28, CD8, CD4, a cytokine receptor, e.g. an interleukin receptor, TNF receptor, or interferon receptor, or a colony stimulating factor receptor e.g. GMCSF. Where desired, the transmembrane component may be the transmembrane domain associated in nature with the co-stimulatory signalling domain as described herein, and may be derived from a tetra-span-transmembrane protein, CD43, CD6, a mannose, IL-7, IL-12 or complement receptor, an integrin-associated protein or a γ-chain associated with a cytokine receptor.

The binding and transmembrane components may be linked directly or, preferably, by a spacer region. The spacer region may be one or more of the regions described above. Where more than one region is present, for example two regions, these are preferably different regions, for example an antibody hinge region linked to all or part of the extracellular region of CD28.

The spacer and transmembrane components are advantageously chosen such that they have free thiol groups thereby providing the chimeric receptor with multimerisation, particularly dimerisation capacity. Receptors of this type, especially dimers, are particularly preferred and include those which have CD28 components, the zeta chain of the natural T-cell receptor, and/or antibody hinge sequences.

The transmembrane component may or may not be naturally linked to the cytoplasmic component to which it is attached either directly or by means of a spacer.

The cytoplasmic signalling components iv) can for example transduce a signal which results in activation of one or more intracellular messenger systems. It is preferred that each of the cytoplasmic components activates a different messenger system. The intracellular messenger systems which may be activated either directly or indirectly include, for example, one or more kinase pathways such as those involving tyrosine kinase, PKC or MAP kinase; G-protein or phospholipase mediated pathways; calcium mediated pathways; and pathways involving synthesis of a cytokine such as an interleukin e.g. IL-2, including NFAT, and cAMP mediated pathways.

At least one of the cytoplasmic signalling components iv) will be a co-stimulatory signalling domain derived from all or part of the receptors particularly described above. In general at least the signalling domain, or the signalling and transmembrane domains may be used, together with the remainder of the receptor as desired, for example to make use of any convenient restriction sites when initially constructing the DNA coding for the receptor.

Thus for example, the co-stimulatory signalling domain may be derived from all or part of a tetra-span-transmembrane protein for example CD9 [Lanza, F et al J. Biol. Chem. 266, 10638-10645 (1991)], especially amino acids from around 36 to around 227; CD37 [Classon, R. et al J. Exp, Med. 172, 1007 (1990)] especially amino acids from around 38 to around 281, CD53 [Angelisova P. et al Immunogenetics 32, 281-285 (1990], especially amino acids from around 37 to 219, CD63 [Metzelaar, M. et al J. Biol. Chem 266, 3239-3245 (1991)], especially amino acids from around 35 to around 237, and CD82 Imai, T. et al J. Immunol. 149, 2879-(1992)], especially amino acids from around 35 to around 267; CD43 [Pallant, A. et al P.N.A.S. 86, 1328-1332 (1989)], especially amino acids from around 262 to around 385 or from around 240 to around 385; CD6 [Aruffo, A. et al J. Exp. Med. 174, 949-952 (1991)], especially amino acids from around 401 to around 644 or from around 374 to around 644; a mannose receptor [Ezekowitz, R et al J.Exp. Med. 172, 1785-1794 (1990)], especially amino acids from around 1394 to around 1438 or from around 1370 to around 1438; an IL-7 receptor a chain (CD127) [Goodwin R. G. Cell 60, 941-951 (1990)], especially amino acids from around 245 to around 439 or from around 220 to around 439; an IL-12 receptor β-chain [Preskey D. et al P.N.A.S. 93, 14002-14007 (1996)], especially amino acids from around 647 to around 862 or from around 623 to around 862; a complement receptor, e.g. CR1 (CD35) [Wong, W., P.N.A.S. 82, 7711-7715 (1985)], especially amino acids from around 1955 to around 1998 or from around 1931 to around 1998, or CR3 and CR3-associated proteins [Messika E. et al, J. Immunol. 154 6563-(1995)]; an integrin-associated protein, such as CD47 [Lindberg, F. et al J. Cell Biol. 123, 485-496 (1993)], including Form 1 and Form 2; or the γ-chain (CD132) of IL-2, IL-4, IL-7, IL-9 or IL-15 receptors [Takeshita, T. et al Science 257, 379-382 (1992)] especially amino acids from around 262 to around 347 or from around 233 to around 347.

It will be appreciated that the above quoted amino acid ranges can be varied as desired, provided the signalling function is retained. Thus for example fragments from within these ranges may be used where this is advantageous in the construction of the DNA coding for the receptor (for example to take into account convenient restriction sites as mentioned above) and/or where this leads to altered and/or improved properties of the receptor, for example to avoid potential glycosylation sites. Thus, in one example DNA coding for a CD9 fragment of amino acid residues 52-227 may be used as an alternative to DNA coding for the full length CD9 signalling domain of amino acid residues 36-227. Fragments of other signalling domains may be advantageously used following the same principles, the size of each fragment depending on the nature of the DNA coding for the parent domain and/or the amino acid sequence of the domain, in particular the location of any restriction and/or glycosylation sites. Such sites can be readily identified in a parent sequence by inspection or other conventional means.

One useful co-stimulatory signalling domain is that derived from all or part of a tetra-span-transmembrane protein, particularly CD9.

In addition to the co-stimulatory signalling domains just described, other suitable cytoplasmic components iv) which may be present in the receptors according to the invention include, for example those derived from the T-cell receptor such as all or part of the zeta, eta or epsilon chain; CD28; the γ chain of a Fc receptor; or signalling components from a cytokine receptor e.g. interleukin, TNF and interferon receptors, a colony stimulating factor receptor e.g. GMCSF, a tyrosine kinase e.g. ZAP-70, fyn, lyk, Itk and syk; an adhesion molecule e.g. LFA-1 and LFA-2, B29, MB-1, CD3 delta, CD3 gamma, CD5 or CD2. The signalling cytoplasmic components are preferably ITAM containing cytoplasmic components

The cytoplasmic signalling components are preferably selected so that they act cooperatively. They may be in any orientation relative to one another. Particularly useful components include all or part of the signalling component of CD28, the zeta chain of the T-cell receptor or the γ chain of FcεR1 or FcγR111 in addition to the co-stimulatory signalling domains described above.

The signal component may be that naturally associated with the binding component or may be derived from other sources.

Examples of suitable signal peptide components i) include immunoglobulin signal sequences.

The signal component, binding component, transmembrane component, and cytoplasmic components are preferably derived from or based on human sequences.

Homologues of the individual components of the chimeric receptor may be used and the invention is to be understood to extend to such use. The term homologue as used herein with respect to a particular nucleotide or amino acid sequence coding for a component of the chimeric receptor represents a corresponding sequence in which one or more nucleotides or amino acids have been added, deleted, substituted or otherwise chemically modified provided always that the homologue retains substantially the same function as the particular component of the chimeric receptor. Homologues may be obtained by standard molecular biology and/or chemistry techniques e.g. by cDNA or gene cloning, or by use of oligonucleotide directed mutagenesis or oligonucleotide directed synthesis techniques or enzymatic cleavage or enzymatic filling in of gapped oligonucleotides.

Fragments of the individual components may also be used wherein one or more nucleotides has been deleted provided that the fragment retains substantially the same function as the starting component of the chimeric receptor.

The DNA for use in this and other aspects of the invention may be obtained from readily available DNA sources using standard molecular biology and/or chemistry procedures, for example by use of oligonucleotide directed mutagenesis or oligonucleotide directed synthesis techniques, enzymatic cleavage or enzymatic filling in of gapped oligonucleotides. Such techniques are described by Maniatis et al in Molecular Cloning, Cold Spring Harbor Laboratory, New York 1989, and in particular in the Examples hereinafter.

The carrier for use in the DNA delivery systems according to the invention may be a vector or other carrier suitable for introduction of the DNA ex-vivo or in-vivo into target cells and/or target host cells. Examples of suitable vectors include viral vectors such as retroviruses, adenoviruses, adenoassociated viruses, EBV, and HSV, and non-viral vectors, such as liposomal vectors and vectors based on DNA condensing agents. Alternatively the carrier may be an antibody. Where appropriate, the vector may additionally include promoter/regulatory sequences and/or replication functions from viruses such as retrovirus LTRs, AAV repeats, SV40 and hCMV promoters and/or enhancers, splicing and polyadenylation signals; EBV and BK virus replication functions. Tissue specific regulatory sequences such as the TCR-α promoter, E-selectin promoter and the CD2 promoter and locus control region may also be used as can inducible promoters, such as hypoxia-induced promoters.

Where two or more DNA molecules are used in the DNA delivery system they may be incorporated into the same or different carriers as described above.

For ex-vivo use, the DNA delivery system of the invention may be introduced into effector cells removed from the target host using methods well known in the art e.g. transfection, transduction, biolistics, protoplast fusion, calcium phosphate precipitated DNA transformation, electroporation, cationic lipofection, or targeted liposomes. The effector cells are then reintroduced into the host using standard techniques.

A wide variety of target hosts may be employed according to the present invention such as, for example, mammals and, especially, humans.

Examples of suitable effector cells include cells associated with the immune system such as lymphocytes e.g. cytotoxic T-lymphocytes, tumour infiltrating lymphocytes, natural killer cells, neutrophils, basophils, eosinophils, mast cells, or T-helper cells; dendritic cells, B-cells, haemoatopaietic stem cells, macrophages, or monocytes. The use of cytotoxic T-lymphocytes is especially preferred. Where macrophages are used, the co-stimulatory signalling domain is preferably derived from all or part of an integrin-associated protein or in particular all or part of a mannose or complement receptor or associated protein. Advantageously in this instance the γ chain of a Fc receptor may be used as a further cytoplasmic signalling component.

The DNA delivery system according to the invention is particularly suitable for in vivo administration. It may be in one preferred example in the form of a targeted delivery system in which the carrier is capable of directing the DNA to a desired effector cell. Particular examples of such targeted delivery systems include targeted-naked DNA, targeted liposomes encapsulating and/or complexed with the DNA, targeted retroviral systems and targeted condensed DNA such as protamine and polylysine condensed DNA.

Targeting systems are well known in the art and include using, for example, antibodies or fragments thereof against cell surface antigens expressed on target cells in vivo such as CD8; CD16; CD4; CD3; selectins e.g. E-selectin; CD5; CD7; CD34; activation antigens e.g. CD69 and IL2R. Alternatively, other receptor—ligand interactions can be used for targeting e.g. CD4 to target HIV_{gp}160—expressing target cells.

In general the use of antibody targeted DNA is preferred, particularly antibody targeted naked DNA, antibody targeted condensed DNA and especially antibody targeted liposomes. Particular types of liposomes which may be used include for example pH-sensitive liposomes where linkers cleaved at low pH may be used to link the antibody to the liposome. Cationic liposomes which fuse with the cell membrane and deliver the recombinant chimeric receptor DNA according to the invention directly into the cytoplasm may also be used. Liposomes for use in the invention may also have hydrophilic groups attached to their surface to increase their circulating half-life such as for example polyethylene glycol polymers. There are many examples in the art of suitable groups for attaching to liposomes or other carriers; see for example International Patent Specifications Nos. WO 88/04924, WO 90/09782, WO 91, 05545, WO 91/05546, WO 93/19738, WO 94/20073 and WO 94/22429. The antibody or other targeting molecule may be linked to the DNA, condensed DNA or liposome using conventional readily available linking groups and reactive functional groups in the antibody e.g. thiols, or amines and the like, and in the DNA or DNA containing materials.

Non-targeted delivery systems may also be used and in these targeted expression of the DNA is advantageous. Targeted expression of the DNA may be achieved for example by using T-cell specific promoter systems such as the zeta promoter and CD2 promoter and locus control region, and the perforin promoter.

The aspect of the invention described above advantageously utilises a single DNA sequence to code for the chimeric receptor. It will be appreciated however that the invention may be extended to DNA delivery systems in which the chimeric receptor is coded for by two or more separate DNA coding sequences. Thus in one example, a first and second separate DNA coding sequence may be present in the delivery system each of which codes for components i) to iv) and optionally v) in the same reading frame as described above but which differ from each other in that the cytoplasmic signalling component iv) is not the same, and always providing of course that at least one of the signalling components is a co-stimulatory signalling domain as described herein. The two DNA coding sequences may each code for more than one signalling component providing that at least one component on the first DNA is different to any other signalling component on the second DNA. As above, the signalling components are advantageously selected to act cooperatively and the remaining components may be any of those previously described for the single DNA embodiment. The binding component iv) coded for by the first DNA will preferably be the same as that coded for by the second DNA. Advantageously the binding component coded by the first DNA will be separated from the transmembrane component by a different spacer region to that coded by the second DNA.

The delivery system may be used ex vivo and in a further aspect the invention provides effector cells transfected with a DNA delivery system according to the invention. The effector cells may be any of those previously described above which are suitable for ex vivo use and are preferably T-cells most preferably cytotoxic T-cells.

The DNA delivery system may take the form of a pharmaceutical composition. It may be a therapeutic or diagnostic composition and may take any suitable form suitable for administration. Preferably it will be in a form suitable for parenteral administration e.g. by injection or infusion, for example by bolus injection or continuous infusion. Where the composition is for injection or infusion, it may take the form of a suspension, solution or emulsion in an oily or aqueous vehicle and it may contain formulatory agents such as suspending, preservative, stabilising and/or dispersing agents. Alternatively, the composition may be in dry form, for reconstitution before use with an appropriate sterile liquid.

If the composition is suitable for oral administration the formulation may contain, in addition to the active ingredient, additives such as: starch—e.g. potato, maize or wheat starch or cellulose—or starch derivatives such as microcrystalline cellulose; silica; various sugars such as lactose; magnesium carbonate and/or calcium phosphate. It is desirable that, if the formulation is for oral administration it will be well tolerated by the patient's digestive system. To this end, it may be desirable to include in the formulation mucus formers and resins. It may also be desirable to improve tolerance by formulating the compositions in a capsule which is insoluble in the gastric juices. It may also be preferable to include the composition in a controlled release formulation.

The DNA delivery system according to the invention is of use in medicine and the invention extends to a method of treatment of a human or animal subject, the method comprising administering to the subject an effective amount of a DNA delivery system described above. The exact amount to be used will depend on the ages and condition of the patient, the nature of the disease or disorder and the route of administration, but may be determined using conventional means, for example by extrapolation of animal experiment derived data. In particular, for ex vivo use the number of transfected effector cells required may be established by ex vivo transfection and re-introduction into an animal model of a range of effector cell numbers. Similarly the quantity of DNA required for in vivo use may be established in animals using a range of DNA concentrations.

The DNA delivery system according to the invention may be useful in the treatment of a number of diseases or disorders. Such diseases or disorders may include those described under the general headings of infectious diseases, e.g. HIV infection; inflammatory disease/autoimmunity e.g. rheumatoid arthritis, osteoarthritis, inflammatory bowel disease; cancer; allergic/atopic diseases e.g. asthma, eczema; congenital e.g. cystic fibrosis, sickle cell anaemia; dermatologic, e.g. psoriasis; neurologic, e.g. multiple sclerosis; transplants e.g. organ transplant rejection, graft-versus-host disease; metabolic/idiopathic disease e.g. diabetes.

DNA coding for a chimeric receptor as described herein also forms a feature of the invention, particularly for use in a delivery system described herein.

The DNA coding for chimeric receptors according to the invention may be assembled from known DNA sequences and expressed in effector cells using the procedures and approaches generally described herein and in our International Patent Specification No. WO 97/23613 and more specifically in the Examples and Figures herein and in WO 97/23613.

The following Examples illustrate the invention:

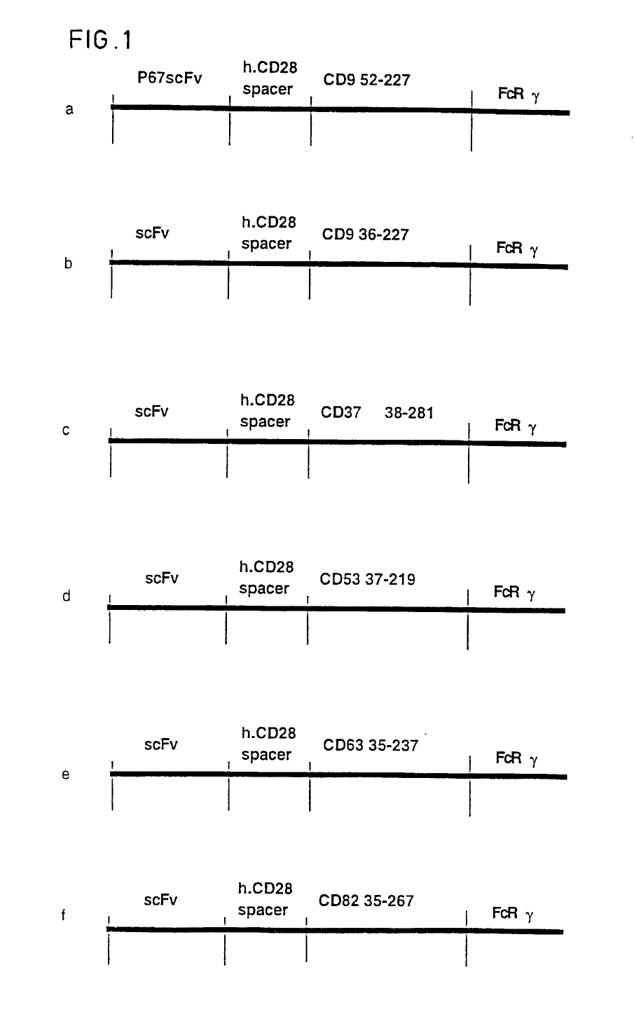

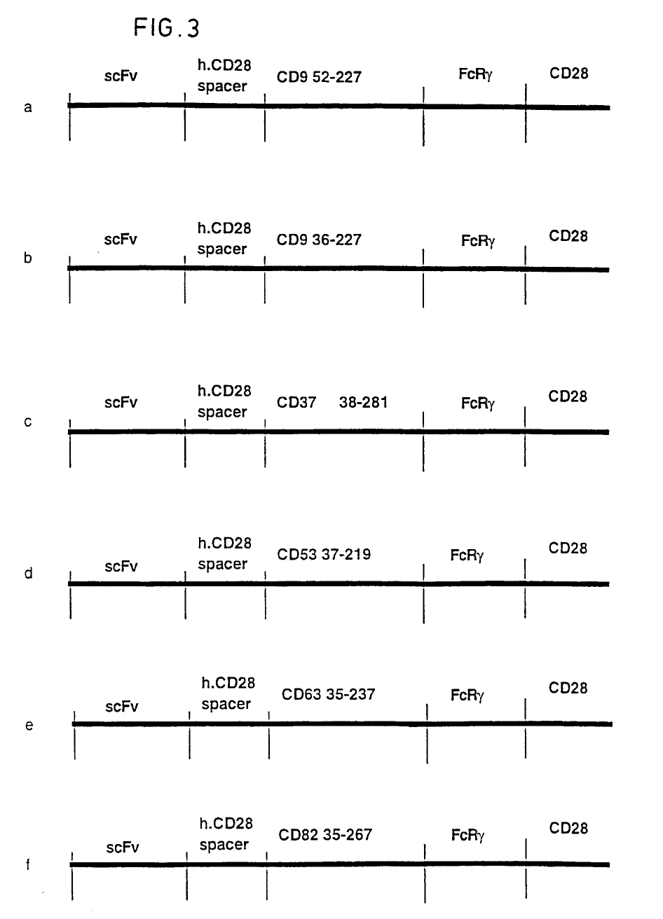

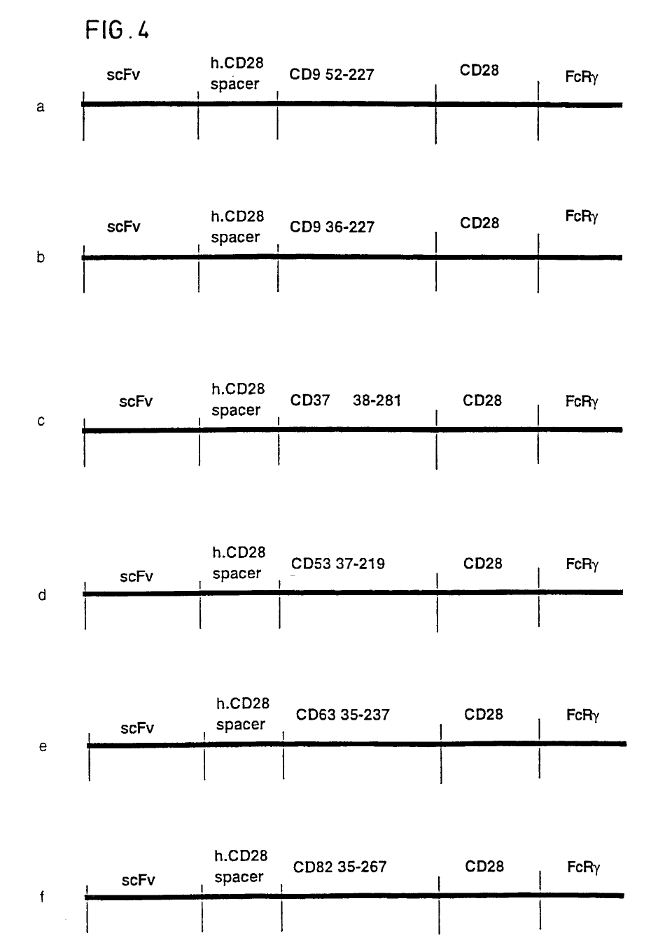

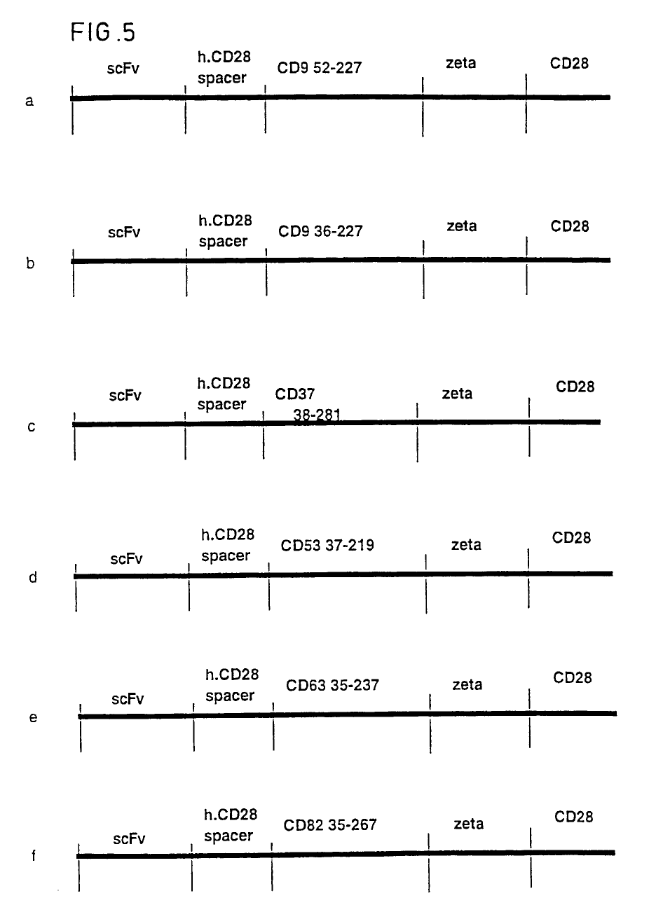

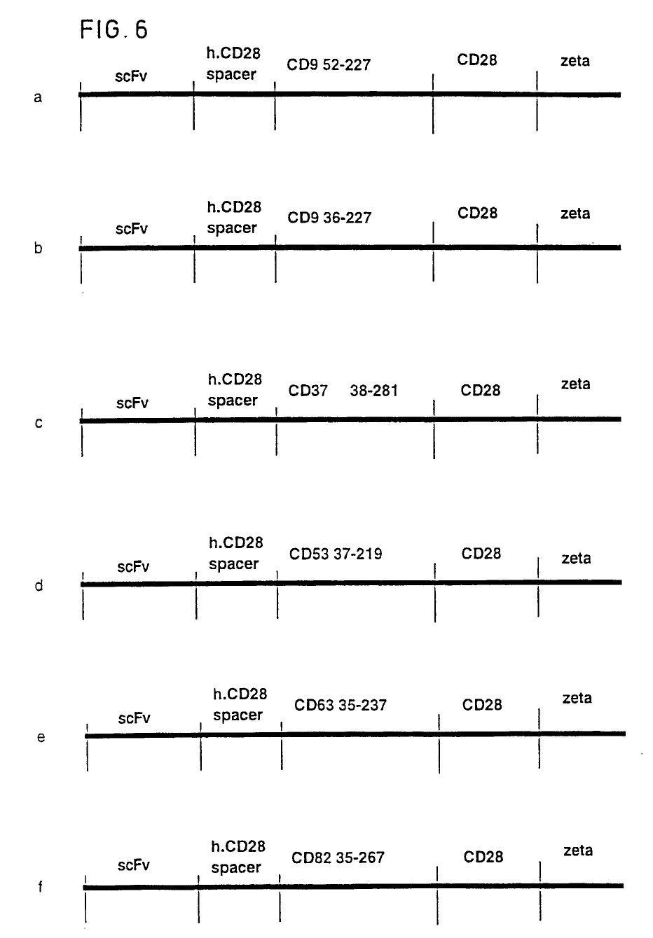

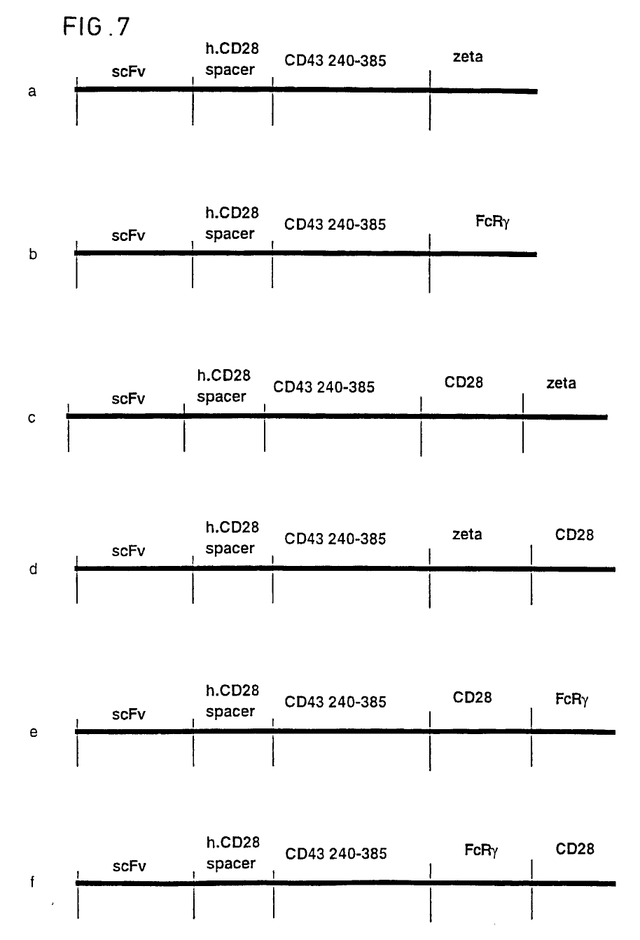

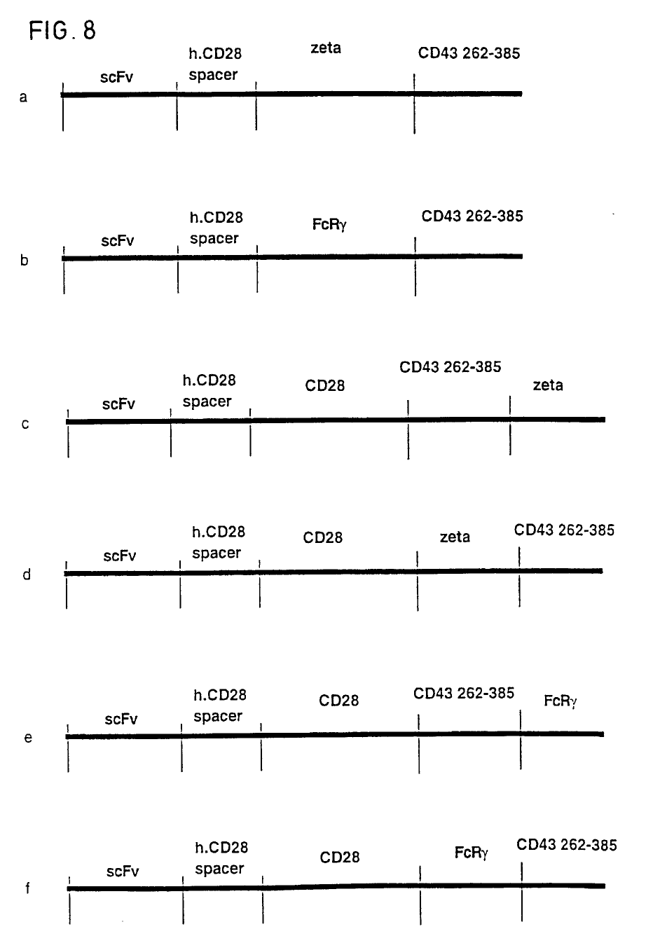

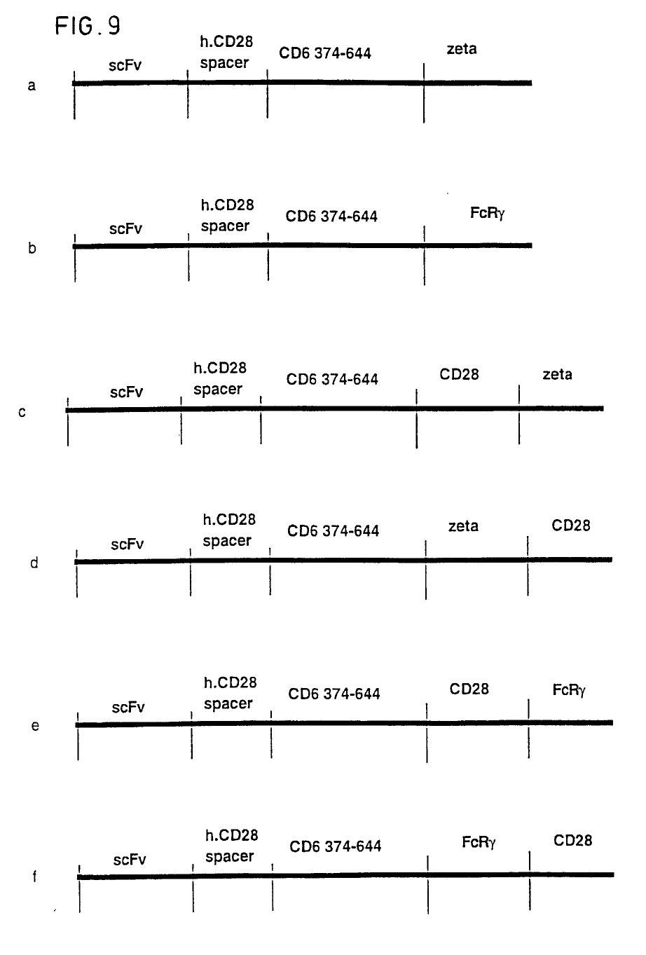

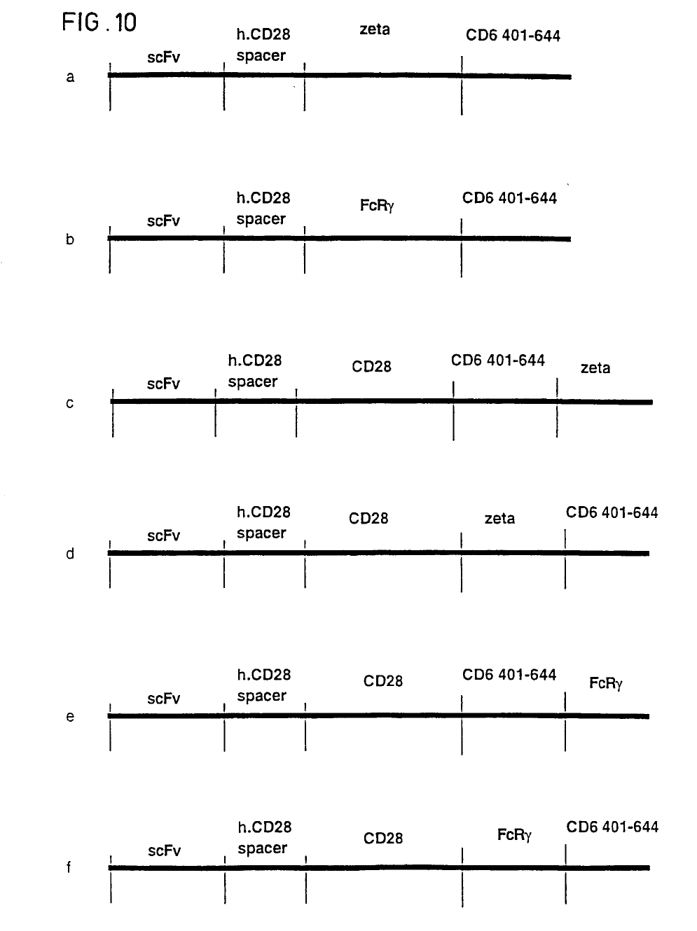

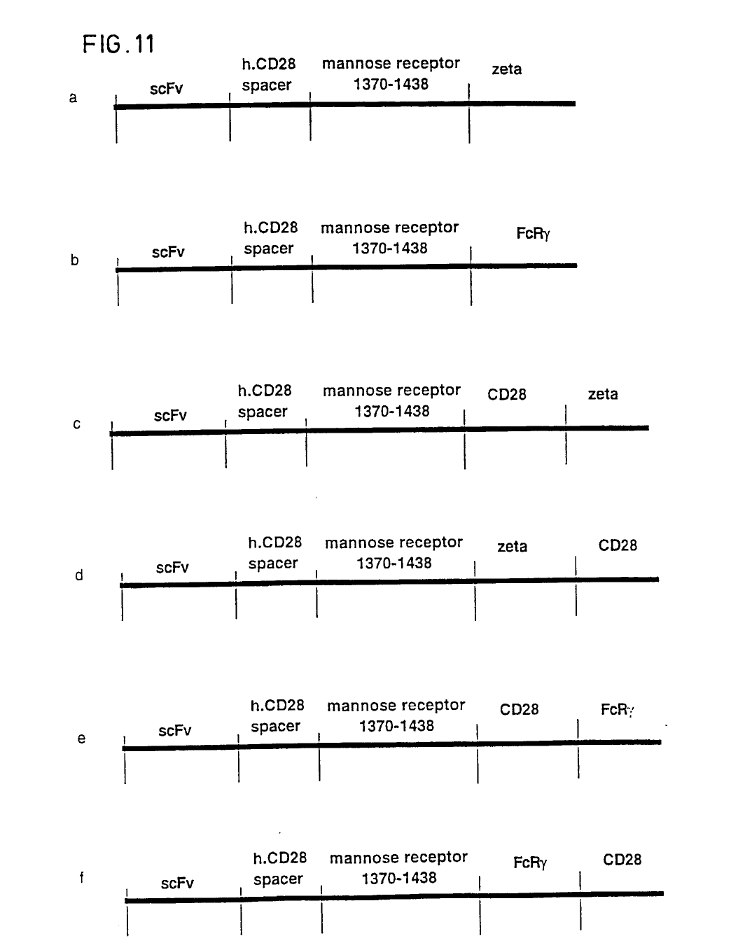

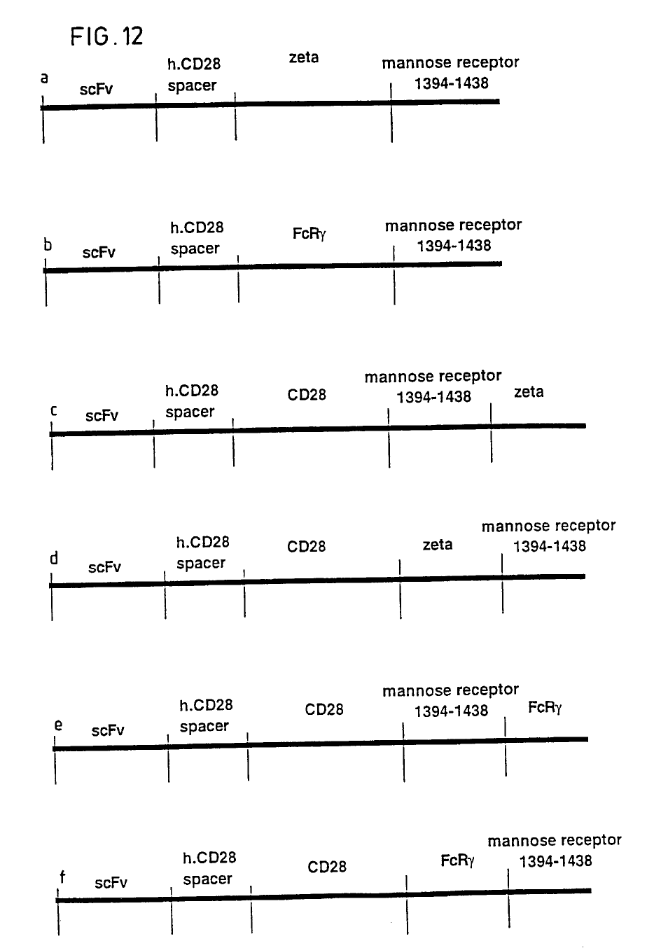

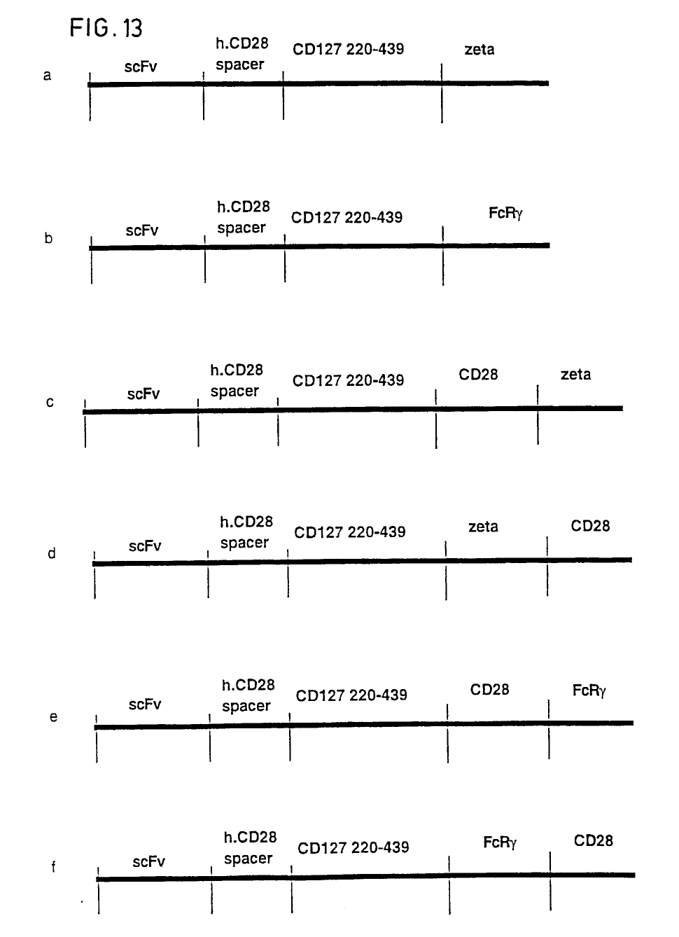

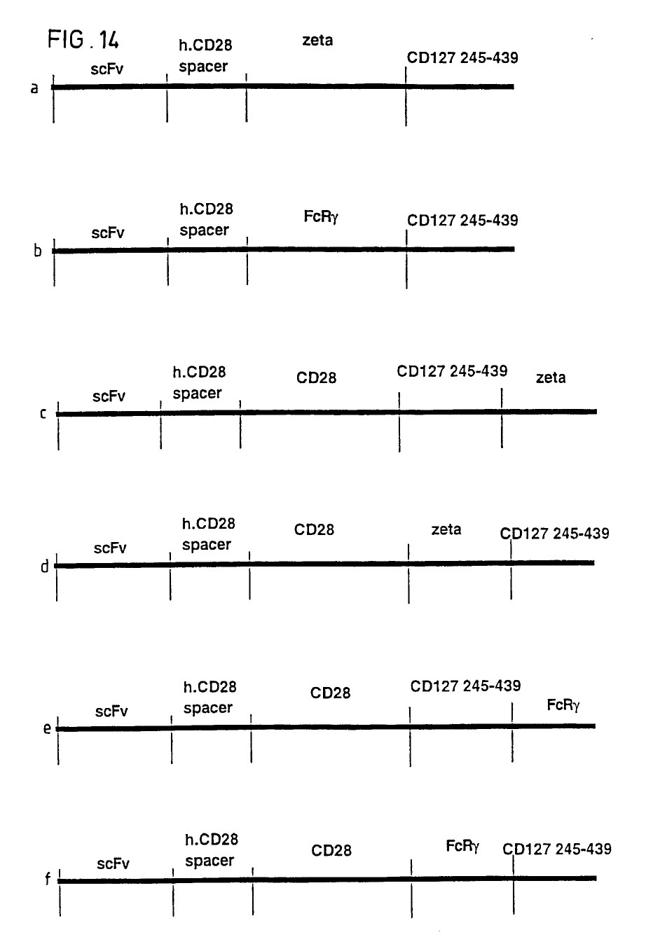

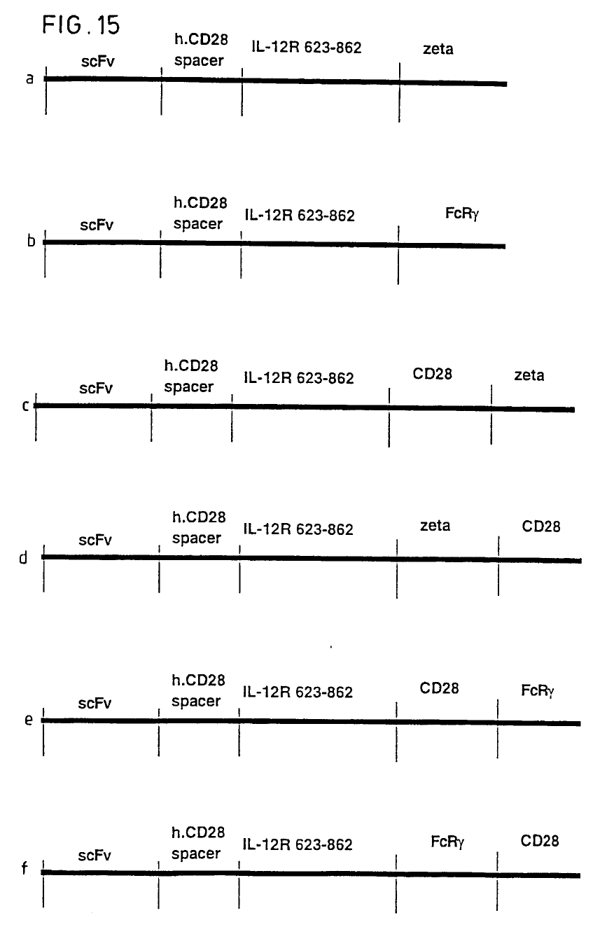

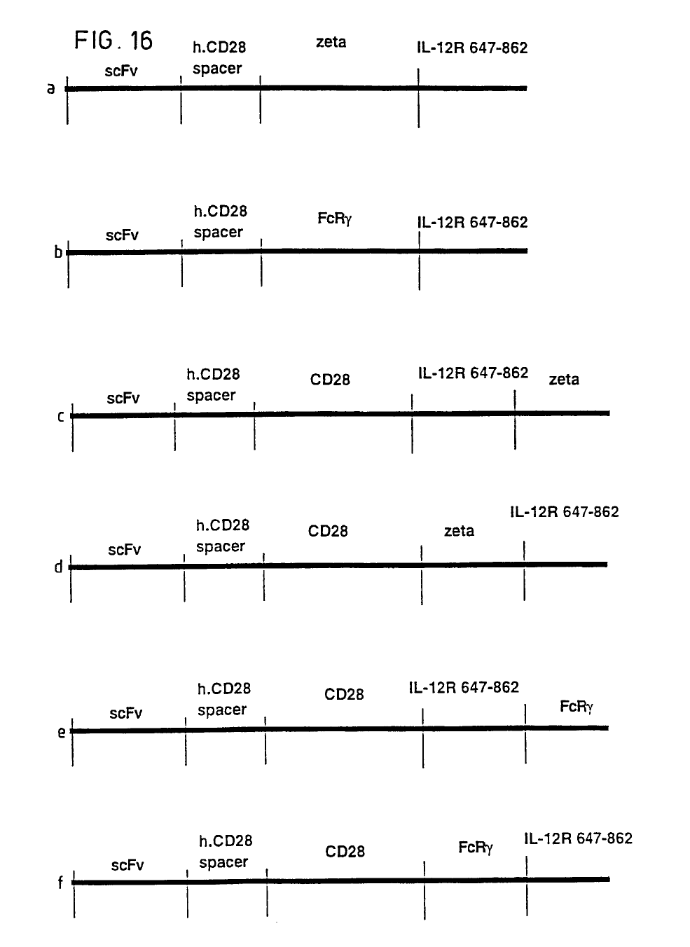

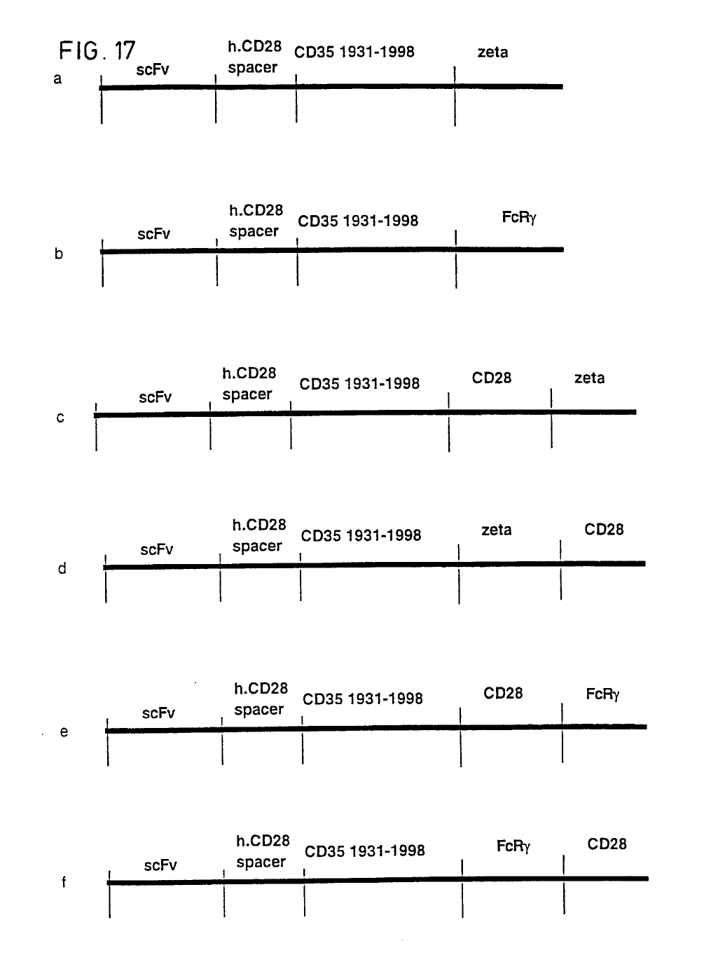

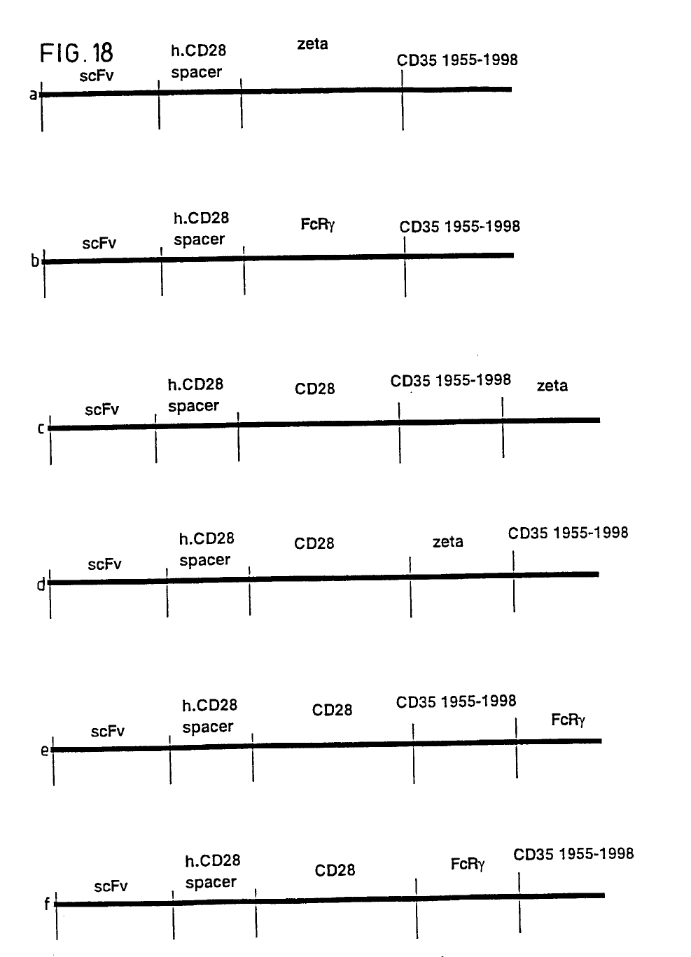

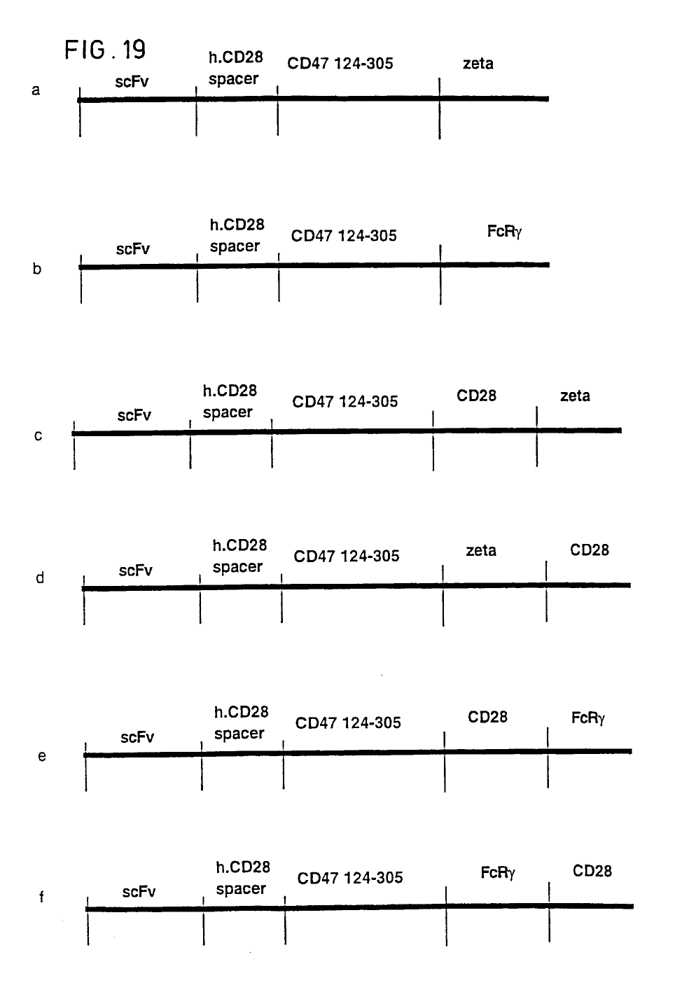

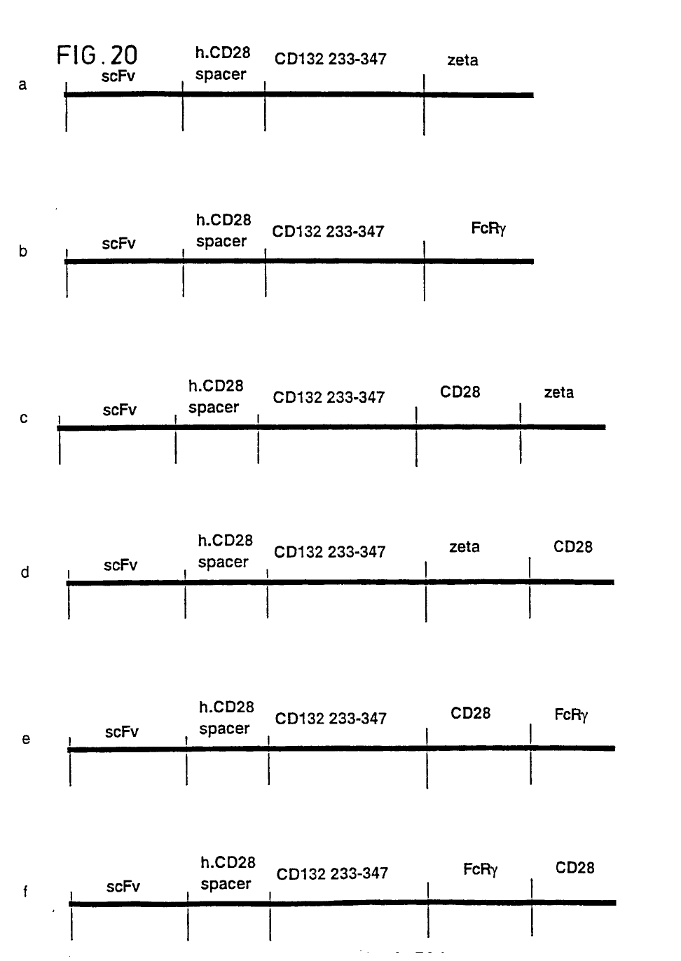

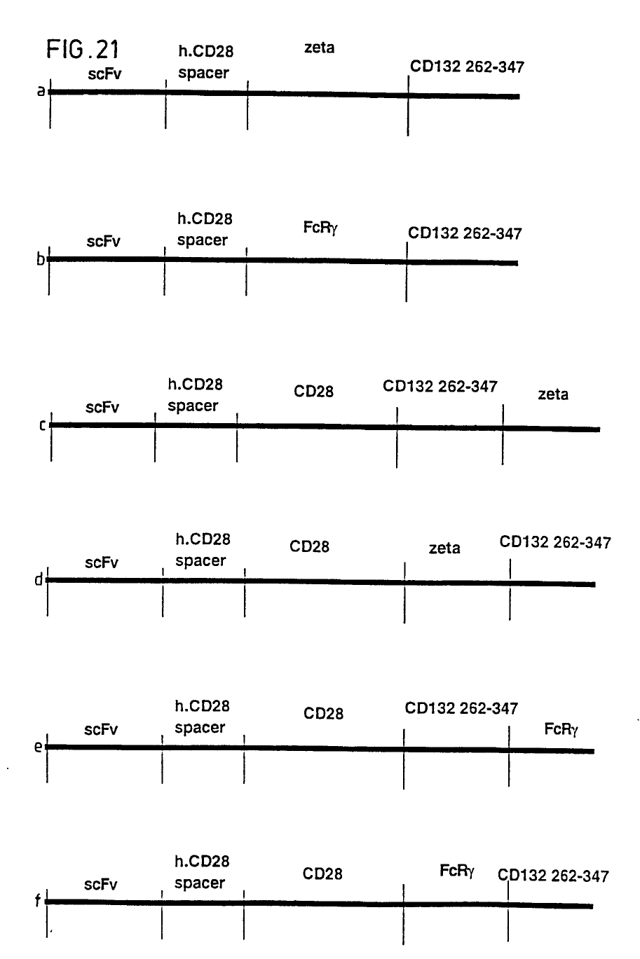

1) Construction of Chimeric Receptor Genes (FIGS. 1-21)

FIGS. 1-21 illustrate chimeric receptor constructs [a-f in each Figure] of the invention.

Each component of these chimeric receptor constructs is either PCR cloned or assembled by standard techniques (PCR protocols, Innis et al, 1990, Academic Press inc.). Each component is flanked by a 2 to 4 amino acid linkage forming a restriction site to allow in frame sub-cloning into pBluescript SK+ (Statagene) in a cassette format similar to that described in Example 1 of our International Patent Specification No. WO 97/23613

Thus, for example, in FIG. 1a the P67 scFv/h.CD28/CD9-FcRγ chimeric receptor consists of a single chain Fv of the engineered human antibody P67.6 specific for human CD33 linked to an extracellular spacer consisting of human IgG1 hinge and part of the extracellular region of human CD28 linked via part of human CD9 to the intracellular domain of the γ chain of human FcεRI.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge and residues 118 to 134 of human CD28. This is linked via residues 52 to 227 of human CD9 (Boucheix et al (1991) J. Biol. Chem. 266, 117-122) to residues 27 to 68 of the γ chain of human FcεRI (Kuster et al (1989) J. Biol. Chem. 255, 6448-6452).

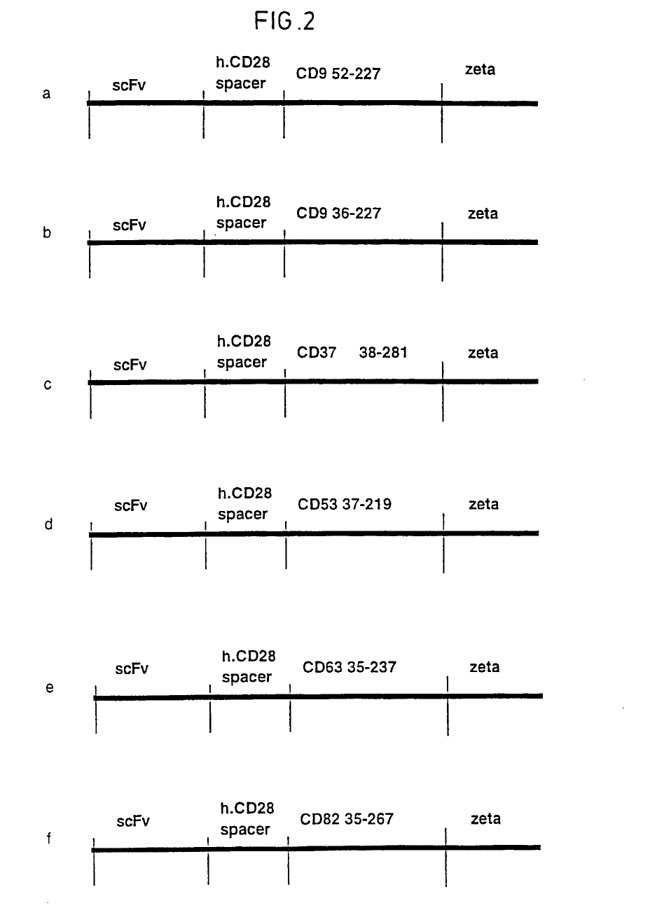

As illustrated in the remainder of FIGS. 1(b-f) and FIGS. 2-21 other receptors according to the invention may be constructed in a similar way using the components shown.

Thus a single chain Fv, such as P67scFv as just described, or hCTMO1scFv as described in Example 1 of WO 97/23613 may be linked to an extracellular spacer, for example consisting of human IgG1 hinge and part of the extracellular region of human CD28 as just described, then linked to the various tansmembrane and intracellular components shown. In each Figure the size of each co-stimulatory domain is shown as CD9 36-227, CD37 38-281, etc. Where in these Figures the size of the particular CD28, zeta and FcRγ component used is not shown, then the same or similarly sized components may be used as described above in the case of FcRγ or as described as described in Example 1 of WO 97/23613 in the cases of CD28 and zeta. Each of these last components may be constructed and incorporated in the receptors according to the present invention in a similar fashion to that described for the receptors described in said Example.

2) Analysis of Chimeric Receptor Constructs Expressed in Jurkat Cells

The following methods, described particularly in relation to the construct in FIG. 1a, may be used to demonstrate the expression and action of receptors according to the invention in transfected cells.

P67scFv/h.CD28/CD9-FcRγ

The full length chimeric receptor construct was sub-cloned from pBluescript into the expression vector pEE6hCMV.ne [Bebbington (1991), Methods 2, 136-145] on a Hind III to EcoR I restriction fragment.

Plasmids were linearised and transfected into Jurkat E6.1 cells (ECACC) by electroporation using a Bio-Rad Gene Pulser using two 1 second pulses of 1000V and 3 μF. Chimeric receptor expressing cell lines were selected in media containing the drug G418 (2 mg/ml) After approximately four weeks cells were analysed for their ability to express surface scFv and to produce IL-2.

Analysis of Surface Expression of scFv

Approximately 5×10⁵ cells were stained with saturating concentrations of fluorescein-conjugated antigen (2 μg.ml). Fluorescence was analysed by a FACScan cytometer (Beckton Dickinson).

Antigen Expressing Cell Stimulation

1×10⁵ Jurkat transfectants were incubated with 1×10⁵ antigen expressing or control non-antigen expressing target cells in a 96 well plate (Falcon) overnight at 37° C./5% CO₂. After 18 to 20 hours cells were centrifuged and supernatant assayed for human IL-2 (Quantikine kit, R & D Systems).

Solid Phase Antigen Stimulation

1×10⁵ Jurkat transfectants were incubated in a 96 well plate (Nunc) previously coated with a saturating concentration of antigen at 37° C./5% CO₂ in non-selective media. After 18 to 20 hours cells were centrifuged and supernatant assayed for human IL-2 (Quantikine kit, R & D Systems).

3) Results

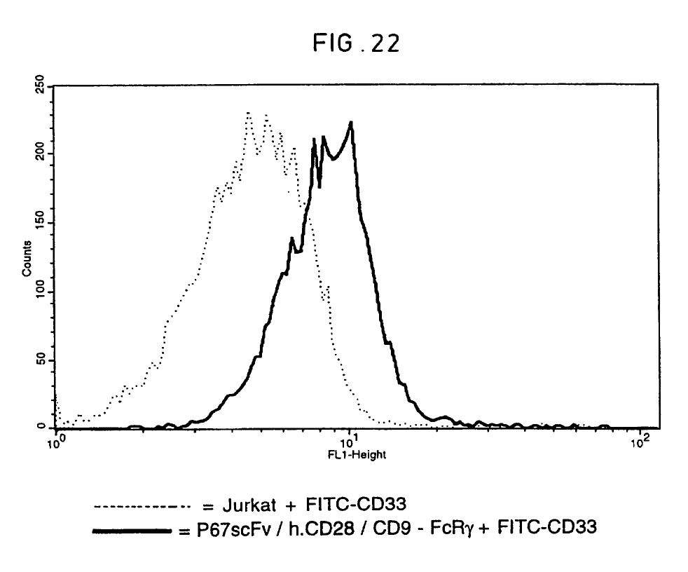

FIG. 22 shows clear surface expression of the P67scFV/h.CD28/CD9-FcRγ chimeric receptor on transfected Jurkat cells as measured with fluorescein-conjugated human CD33.

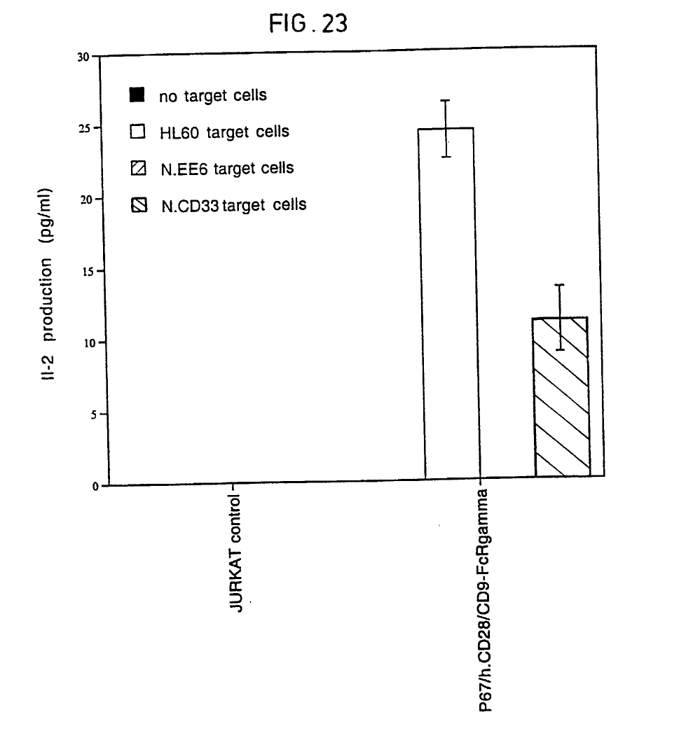

FIG. 23 shows antigen specific IL-2 production by Jurkat cells transfected with the P67scFV/h.CD28/CD9-FcRγ chimeric receptor compared to untransfected Jurkat cells when stimulated with antigen positive target cells [HL60 and N.CD33 (the mouse myeloma NSO expressing human CD33)] as compared to no target cells or antigen negative cells [N.EE6 (the mouse myeloma NSO expressing a control plasmid)]. The figure shows that in the presence of the antigen (CD33) positive cells transfected Jurkat cells expressing the chimeric receptor were stimulated to produce IL-2. The same cells failed to produce IL-2 in the presence of cells not presenting the antigen. In each instance, non-transfected Jurkat control cells also failed to respond and produced no measurable quantities of IL-2.

1. A method of activating a cell as a result of one type of extracellular interaction between said first cell and a molecule associated with a second target cell characterised in that said first cell is provided with a DNA delivery system comprising DNA coding for one or more recombinant chimeric receptors comprising two or more different cytoplasmic signalling components, wherein said cytoplasmic components are not naturally linked, and at least one is derived from a membrane spanning polypeptide, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor.

2. A method according to claim 1 wherein the cytoplasmic signalling components are capable of acting together cooperatively.

3. A method according to claim 1 or claim 2 wherein said DNA additionally codes for signal peptide, binding and/or transmembrane components of said one or more chimeric receptors, wherein the binding component is capable of recognising a cell surface molecule on a target cell.

4. A method according to claim 3 wherein the signal peptide, transmembrane and cytoplasmic signalling components and all or part of the binding component are coded for by a single DNA coding sequence.

5. A method according to claim 3 wherein each cytoplasmic signalling component is coded for by a separate DNA coding sequence, each DNA sequence additionally coding for a signal peptide, a transmembrane component and all or part of a binding component.

6. A method according to claim 4 or claim 5 wherein said DNA codes for part of said binding component and an additional separate DNA coding sequence codes for the remainder of the binding component.

7. A method according to claim 5 or claim 6 wherein the binding component coded for by one DNA sequence is capable of participating in the same type of extracellular binding event as the binding component coded for by any other DNA sequence.

8. A method according to claim 7 wherein each binding component binds to the same molecule associated with the target cell.

9. A method according to claim 8 wherein each binding component is the same.

10. A method according to any one of claims 1 to 9 wherein the one or more recombinant chimeric receptors are capable of recognising a viral or cell surface molecule on a target cell.

11. A DNA delivery system comprising DNA in association with a carrier said DNA coding for a recombinant chimeric receptor capable of one type of extracellular interaction and comprising two or more different cytoplasmic signalling components which are not naturally linked, and wherein at least one of said cytoplasmic components is derived from a membrane spanning polypeptide, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor.

12. A DNA delivery system comprising DNA in association with a carrier said DNA coding for two or more recombinant chimeric receptors each capable of the same one type of extracellular interaction and wherein each of said receptors comprises one or more different cytoplasmic signalling components which are not naturally linked, and wherein at least one of said cytoplasmic components is derived from a membrane spanning polypeptide, characterised in that at least one of said cytoplasmic signalling components is a co-stimulatory signalling domain derived from all or part of a tetra-span-transmembrane protein, CD43, CD6, or a mannose, IL-7, IL-12 or complement receptor, an integrin-associated protein, or a γ-chain associated with a cytokine receptor.

13. A DNA delivery system according to claim 11 wherein said DNA codes in reading frame for:
i) a signal peptide component;
ii) a binding component capable of recognising a cell surface molecule on a target cell;
iii) a transmembrane component;
iv) two or more different cytoplasmic signalling components which are not naturally linked, and wherein at least one of said cytoplasmic components is derived from a membrane spanning polypeptide; and optionally
v) one or more spacer regions linking any two or more of said i) to iv) components, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor.

14. A DNA delivery system according to claim 11 wherein said DNA comprises 1) a first DNA which codes in reading frame for:
i) a signal peptide component;
ii) one or part of a binding component;
iii) a transmembrane component;
iv) two or more cytoplasmic signalling components which are not naturally linked, and wherein at least one of said cytoplasmic components is derived from a membrane spanning polypeptide; and optionally
v) one or more spacer regions linking any two or more of said i) to iv) components, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor; and 2) a second separate DNA which codes in reading frame for a signal peptide component and a further part of the binding component ii) coded for by said first DNA, such that the binding component parts together are capable of recognising a cell surface molecule on a target cell.

15. A DNA delivery system according to claim 12 wherein said DNA comprises a first and a second separate DNA each of which codes in reading frame for:
i) a signal peptide component;
ii) a binding component capable of recognising a cell surface molecule on a target cell;
iii) a transmembrane component;
iv) one or more different cytoplasmic signalling components which are not naturally linked, and wherein at least one of said cytoplasmic components is derived from a membrane spanning polypeptide; and optionally
v) one or more spacer regions linking any two or more of said i) to iv) components, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor; provided that said first DNA codes for at least one signalling component iv) that is not coded for by said second DNA.

16. A DNA delivery system according to claim 12 wherein said DNA comprises 1) a first and a second separate DNA each of which codes in reading frame for:
i) a signal peptide component;
ii) one part of a binding component;
iii) a transmembrane component;
iv) one or more different cytoplasmic signalling components which are not naturally linked, and wherein at least one of said cytoplasmic components is derived from a membrane spanning polypeptide; and optionally
v) one or more spacer regions linking any two or more of said i) to iv) components, characterised in that at least one of said cytoplasmic signalling components is a co-stimulatory signalling domain derived from all or part of a tetra-span-transmembrane protein, CD43, CD6, a mannose, IL-7, IL-12 or complement receptor, an integrin-associated protein, or a γ-chain associated with a cytokine receptor; provided that said first DNA codes for at least one signalling component iv) that is not coded for by said second DNA; and 2) a separate third and fourth DNA each of which codes in reading frame for a signal peptide component and a further part of the binding component ii) coded for by said first and second DNA respectively, such that the binding component parts together provided by the first and third DNA and together provided by the second and fourth DNA are each capable of recognising a cell surface molecule on a target cell.

17. A DNA delivery system according to claims 13 to 16 wherein each signal peptide component is an immunoglobulin signal sequence.

18. A DNA delivery system according to claims 15 to 17 wherein the binding component coded for by said first DNA is the same as the binding component coded for by said second DNA.

19. A DNA delivery system according to claims 13 to 18 wherein the binding component is an antibody or an antigen binding fragment thereof.

20. A DNA delivery system according to claim 19 wherein the antibody or fragment thereof is an engineered human antibody or antigen binding fragment thereof.

21. A DNA delivery system according to claims 18 to 20 wherein the binding component is a single chain Fv fragment.

22. A DNA delivery system according to claims 18 to 20 wherein the binding component is a Fab′ fragment.

23. A DNA delivery system according to any one of claims 13 to 22 wherein the transmembrane component is derived from all or part of the alpha, beta or zeta chain of the T-cell receptor, CD28, CD8, CD4, a cytokine receptor, a colony stimulating factor receptor, a tetra-span-transmembrane protein, CD43, CD6, a mannose, IL-7, IL-12 or complement receptor, an integrin-associated protein, or a γ-chain associated with a cytokine receptor.

24. A DNA delivery system according to claim 23 wherein the transmembrane component is derived from all or part of CD28.

25. A DNA delivery system according to any one of claims 11 to 24 wherein the costimulatory signalling domain is derived from all or part of CD9, CD37, CD53, CD82, CD43, CD6, CD127, an IL-12 receptor β-chain, CD35, CD3 and CR3-associated proteins, CD47 or the γ-chain of IL-2, IL-4, IL-7, IL-9 or IL-15 receptors.

26. A DNA delivery system according to claim 25 wherein the costimulatory domain is derived from all or part of CD9.

27. A DNA delivery system according to any one of claims 13 to 26 wherein at least one other of the cytoplasmic signalling components is derived from all or part of the cytoplasmic domains of a zeta, eta or epsilon chain of the T-cell receptor, CD28, the γ chain of a Fc receptor, a cytokine receptor, a colony stimulating factor receptor, a tyrosine kinase or an adhesion molecule, B29, MB-1, CD3 delta, CD3 gamma, CD5 or CD2.

28. A DNA delivery system according to claim 27 wherein the cytoplasmic signalling components are derived from all or part of CD28, the zeta chain of the T-cell receptor and/or the γ chain of FcεR1 or FcγR111

29. A DNA delivery system according to any one of claims 11 to 28 wherein the cytoplasmic signalling components are in any orientation relative to one another.

30. A DNA delivery system according to any one of claims 13 to 29 wherein said DNA coding for components i) to iv) additionally codes for one or more spacer regions linking the binding component ii) and the transmembrane component iii).

31. A DNA delivery system according to claim 30 wherein two or more different spacer regions link the binding component ii) and the transmembrane component iii).

32. A DNA delivery system according to claims 30 or claim 31 wherein the spacer region is selected to provide one or more free thiol groups.

33. A DNA delivery system according to claims 30 to 32 wherein the spacer region is derived from all or part of the extracellular region of CD8, CD4 or CD28.

34. A DNA delivery system according to claims 30 or claim 32 wherein the spacer region is all or part of an antibody constant region.

35. A DNA delivery system according to claims 30 to 32 wherein the spacer region is derived from all or part of an antibody hinge region linked to all or part of the extracellular region of CD28.

36. A DNA delivery system according to any one of claims 11 to 35 wherein the carrier is a viral vector or a non-viral vector.

37. A DNA delivery system according to claim 36 wherein the non-viral vector is a liposomal vector.

38. A DNA delivery system according to claim 37 wherein the carrier is a targeted non-viral vector.

39. A DNA delivery system according to claim 38 wherein the targeted vector is an antibody targeted liposome.

40. A DNA delivery system according to claim 38 wherein the targeted vector is an antibody targeted condensed DNA.

41. A DNA delivery system according to claim 40 wherein the targeted vector is an antibody targeted protamine or polylysine condensed DNA.

42. A DNA delivery system according to claim 38 wherein the targeted vector is antibody targeted naked DNA.

43. A DNA delivery system according to claims 39 to 42 wherein the antibody is a whole antibody or an antigen binding fragment thereof.

44. A DNA delivery system according to claim 43 wherein the antibody is an engineered human antibody or an antigen binding fragment thereof.

45. An effector cell transfected with a DNA delivery system according to any one of claims 1 to 444.

46. An effector cell according to claim 45 which is a lymphocyte, a dendritic cell, a B-cell, a haematopoietic stem cell, a macrophage, a monocyte or a NK cell.

47. An effector cell according to claim 46 which is a cytotoxic T-lymphocyte.

48. A DNA delivery system according to any one of claims 11 to 47 for use in the treatment of infectious disease, inflammatory disease, cancer, allergic/atopic disease, congenital disease, dermatologic disease, neurologic disease, transplants and metabolic/idiopathic disease.

49. A DNA delivery system according to claim 48 for use in the treatment of rheumatoid arthritis, osteoarthritis, inflammatory bowel disease, asthma, eczema, cystic fibrosis, sickle cell anaemia, psoriasis, multiple sclerosis, organ or tissue transplant rejection, graft-versus-host disease or diabetes.

50. A pharmaceutical composition comprising a DNA delivery system according to any one of claims 11 to 44 together with one or more formulatory agents.

51. A pharmaceutical composition according to claim 50 wherein the formulatory agent is a suspending, preservative, stabilising and/or dispersing agent.

52. DNA coding for a recombinant chimeric receptor for use in a delivery system according to any one of claims 11 to 44.
